(19) United States
(12) Patent Application Publication
(10) Pub. No.: US 2023/0106872 A1
(43) Pub. Date: Apr. 6, 2023

(54) ATHLETIC PERFORMANCE ESTIMATION APPARATUS, ATHLETIC PERFORMANCE ESTIMATION METHOD, AND PROGRAM

(71) Applicant: NIPPON TELEGRAPH AND TELEPHONE CORPORATION, Tokyo (JP)

(72) Inventors: Naoki SAIJO, Tokyo (JP); Hsin-I LIAO, Tokyo (JP)

(73) Assignee: NIPPON TELEGRAPH AND TELEPHONE CORPORATION, Tokyo (JP)

(21) Appl. No.: 17/914,279

(22) PCT Filed: Mar. 30, 2020
(86) PCT No.: PCT/JP2020/014494

§ 371 Date: Sep. 23, 2022

(51) Int. Cl. A61B 5/16; A61B 3/113

(52) U.S. Cl. CPC A61B 5/163; A61B 3/113; A61B 2503/10

(57) ABSTRACT

An athletic performance estimation apparatus evaluates athletic performance from the movement of eyes when observing the movement of a target. A control circuitry (11) selects a measurement condition. A video presentation circuitry (12) presents video according to the measurement condition to an observed person. An eyeball movement measurement circuitry (13) measures the movement of eyes of the observed person who is observing the movement of a target. An analysis circuitry (14) obtains a feature value based on an eyeball movement, from time series information on the movement of the eyes. A normalization circuitry (15) integrates the obtained feature value with a feature value obtained under a different measurement condition and performs normalization. An estimation circuitry (16) estimates the athletic performance of the observed person from a normalized feature value.

### TECHNICAL FIELD

The present invention relates to a technique for estimating the athletic performance of an observed person.

### BACKGROUND ART

In Patent Literature 1, a technique is disclosed in which the extent of an attention range (focus range) is estimated from the movement of the eyes of an observed person who is exercising and based on it, the athletic performance such as reaction speed and reaction accuracy of the observed person is estimated. In Patent Literature 1, in estimating the extent of the attention range, both the property that information on the dynamic change of the eyes of the observed person (e.g., microsaccades) and the extent of the attention range of the observed person correlate with each other and the property that there is a correlation between the extent of the attention range and the reaction speed and reaction accuracy of the observed person are used.

### PRIOR ART LITERATURE

### Patent Literature

Patent Literature 1: Japanese Patent Application Laid-Open No. 2019-30491

### SUMMARY OF THE INVENTION

### Problems to be Solved by the Invention

The technique disclosed in Patent Literature 1 is to estimate the athletic performance of the observed person by using the subtle movement of eyeballs that unconsciously occurs in gazing at an object. However, in actual sport environments, eyes follow a moving object in many scenes. Therefore, it is difficult to measure in an actual environment the subtle movement of eyeballs that occurs in gazing at one point and to evaluate the athletic performance.

It is an object of the present invention in view of the above technical problem to provide a technique capable of evaluating athletic performance from the movement of eyes when observing the movement of a target.

### Means to Solve the Problems

In order to solve the above problem, an athletic performance estimation apparatus according to one aspect of the present invention includes: an analysis circuitry that obtains a feature value based on eyeball movement of an observed person who is observing movement of a target; and an estimation circuitry that estimates athletic performance of the observed person from the feature value obtained from the observed person, based on a predetermined relationship between the feature value based on the eyeball movement and a level of athletic performance.

### Effects of the Invention

According to the invention, it is possible to evaluate athletic performance from the movement of eyes when observing the movement of a target.

### BRIEF DESCRIPTION OF THE DRAWINGS

FIG. 1A is a diagram for explaining an experimental result as a background of the present invention and shows a result of an experiment in which a subject is a skilled person.

FIG. 1B is a result of an experiment in which a subject is an unskilled person.

FIG. 2 is a diagram illustrating a functional configuration of an athletic performance estimation apparatus of a first embodiment.

FIG. 3 is a diagram illustrating a procedure of an athletic performance estimation method of the first embodiment.

FIG. 4 is a diagram illustrating a functional configuration of an athletic performance estimation apparatus of a second embodiment.

FIG. 5 is a diagram illustrating a procedure of an athletic performance estimation method of the second embodiment.

FIG. 6 is a diagram illustrating a functional configuration of a computer.

### DETAILED DESCRIPTION OF THE EMBODIMENTS

Hereinafter, embodiments of the invention will be described in detail. It should be noted that the components having the same function in the drawings are denoted by the same numerals to omit redundant explanations.

[Experimental Result]

First, an experimental result as a background of the invention will be described.

In this experiment, a task was executed, with respect to a specific sport, where presenting a video, in which the observation target (e.g., a thing such as a ball or the like, or a person such as an opponent or the like; hereinafter, simply referred to as “target”) is about to move, to a subject and making the subject predict which direction the target will move. At this time, the eyeball movement of the subject watching the video was obtained by using a measurement device such as an eye tracker. More specifically, at the penalty kick scene in soccer, the subject was made to watch the motion of the kicker, where a kicker ran from a position in the right half of the screen toward the ball placed at the center of the screen and was about to kick the ball, by the video of about two seconds long shot from the goal keeper viewpoint, and made to predict whether the kicked ball went to right or left. On this occasion, the screen was darkened after the moment of kicking. That is, the subject predicted the direction in which the ball went, from the motion to kick, without watching the scene where the ball actually went.

The measurement device obtained the direction, angular velocity, and angular acceleration of the eyeballs of the subject at each time and generated time series information on the movement of eyes. Based on this time series information, the start time and magnitude (amplitude) of saccadic eye movement (saccades) were determined. The saccades include a micro saccadic eye movement (microsaccades) that has the amplitude of about 1° and only unconsciously occurs, and a saccadic eye movement that has a larger amplitude than that and can occur consciously. Here, the former microsaccades was to be measured. More specifically, from the movement of eyes at each time which was obtained by the measurement device, the eyeball movement whose maximum angular velocity and maximum angular acceleration were within predetermined reference values was detected as microsaccades, and the time and magnitude thereof were extracted.

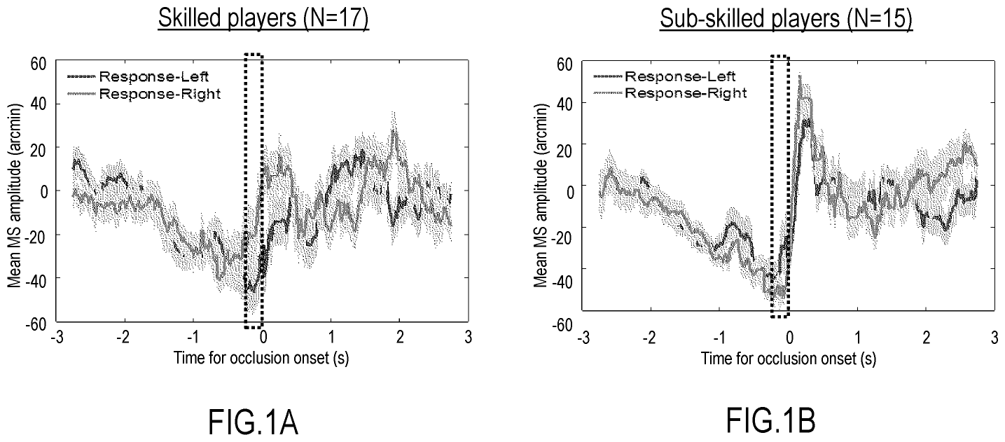

FIG. 1 illustrates an analysis result of the characteristics of eyeball movement (microsaccade information) in the experiment described above. A graph in FIG. 1A shows mean values of microsaccade information that was obtained from skilled persons in soccer (Skilled players); and a graph in FIG. 1B shows mean values of microsaccade information that was obtained from unskilled persons (Sub-skilled players). A horizontal axis indicates time of microsaccades occurrence. The time of 0 seconds indicates a video end time (that is, the time when a subject predicted a movement). In the above experiment, the video end time is a moment of kicking and therefore, the subject was watching the video of a motion before the kicking for a time period of negative time and was watching a darkened screen for a time period of positive time. A vertical axis indicates the amplitude of microsaccades. Here the amplitude is represented by angle (arcmin); positive values correspond to the right direction and negative values correspond to the left direction. In both graphs, darker lines (Response-Left) indicate data of the subjects when they were watching video in which a ball went to the left after the prediction time (correct answer was the left); and lighter lines (Response-Right) indicate data of the subjects when they were watching video in which a ball went to the right after the prediction time (correct answer was the right).

First, for both the skilled persons and unskilled persons, there was found a tendency of a decrease in the amplitude of microsaccades in the negative direction from the start of the kicker (time of around −2 seconds) to the moment of kicking (time of 0 seconds). This is considered to be caused by the reasons: since the kicker ran toward the center of the screen from a position on the right side of the center of the screen before the ball was kicked, the attention of the subject tended to be directed to the left direction; and the eyeball movement of tracking in the left direction occurred in order to follow the kicker.

Next, attention is given to a time period immediately before the moment of kicking (area surrounded by a dotted square, from around −200 milliseconds to 0 seconds). In a result for the skilled persons (FIG. 1A), the amplitude greatly leaned to the positive when the correct answer was the right and remained negative when the correct answer was the left; thus, there was seen a significant difference in its movement according to whether the direction of the correct answer was the left or the right. On the other hand, in a result for the unskilled persons (FIG. 1B), the amplitude was about the same whether the correct answer is right or left; and there was not seen a difference by the directions of the correct answer. That is, in the result of the skilled persons, in a time period immediately before the prediction time, change in the amplitude was observed in the direction corresponding to the predicted direction. On the other hand, in the result of the unskilled persons, such a change was not seen.

Here, it is considered that the change in the amplitude obtained in the above experiment includes the one corresponding to the direction in which the ball was predicted to go and the one corresponding to the direction in which the kicker moved. However, it is difficult to separate them. Hence, suppose a video obtained by horizontally flipping the video used in the above experiment is presented to the same subject and the same task is performed to obtain similar experiment results. Then, in the flipped video, the kicker runs from a position on the left of the center of the screen toward the center of the screen and therefore, it is expected that there appears the tendency of the amplitude of microsaccades increasing in the positive direction from the kicker's starting to move (time of around −2 seconds) to the moment of kicking (time of 0 seconds). On the other hand, a change in the amplitude corresponding to a direction in which the ball is predicted to go should occur in a manner similar to the one when unflipped video is presented. Thus, when data obtained by presenting unflipped video and data obtained by presenting flipped video are integrated, a change in the amplitude related to the motion of the kicker is canceled and only the change in the amplitude related to the direction in which the ball is predicted to go can be extracted. The change in the amplitude thus extracted indicates that there is a tendency in the skilled persons, that their eyeballs precedingly move in a direction in which they potentially (unconsciously) predict the ball to go. In other words, it can be said to indicate the presence/absence of a potential prediction ability of the subject (that the skilled persons are assumed to have a higher prediction ability).

The present invention utilizes the finding of such a correlation and thereby estimates the athletic performance (skill level, or the presence/absence of potential prediction ability) from the movement of the eyes of the observed person performing the task of predicting the movement of a target.

### First Embodiment

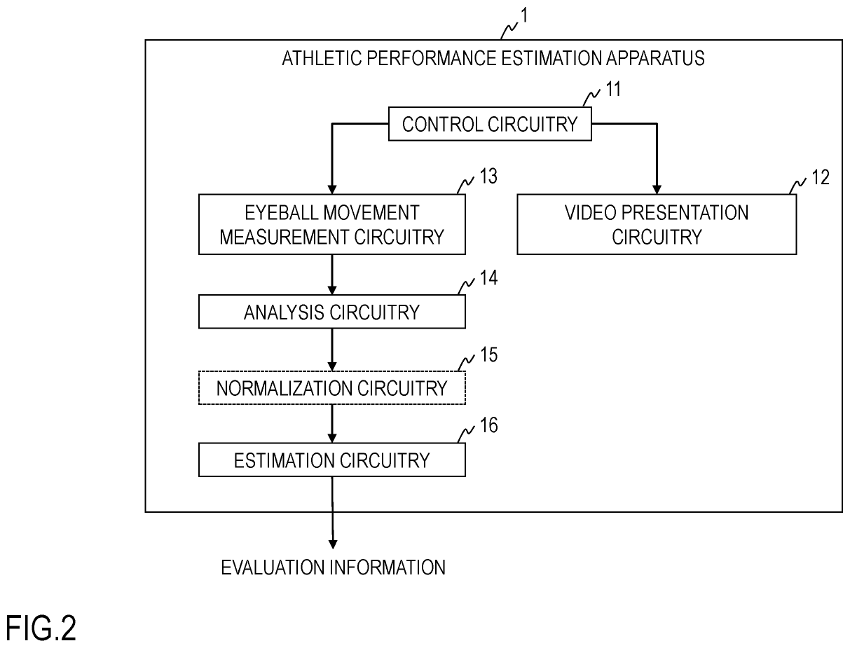

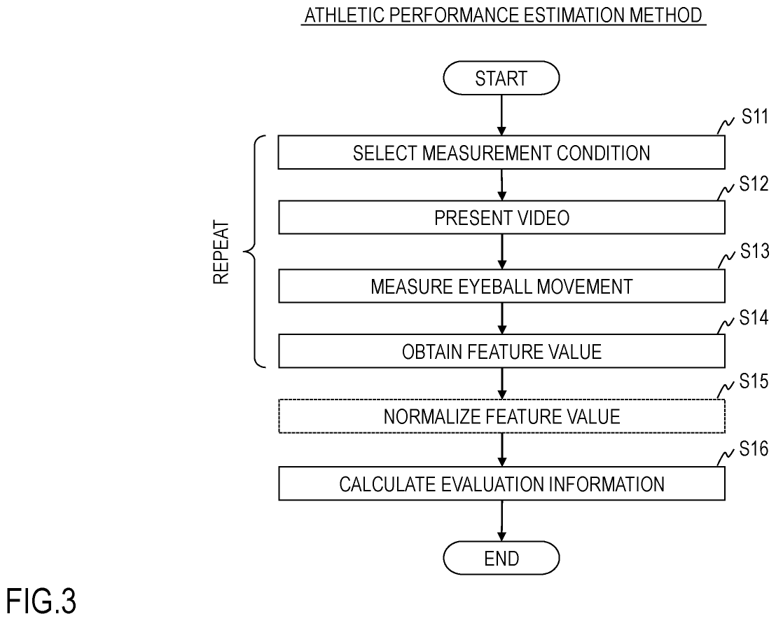

The first embodiment of the present invention is an athletic performance estimation apparatus and method with which preliminarily prepared video is presented to an observed person (subject) and from the movement of the eyes of the observed person who is watching the video, the athletic performance of the observed person is estimated. The athletic performance estimation apparatus 1 of the first embodiment includes, as illustrated in FIG. 2, for example, a control circuitry 11, a video presentation circuitry 12, an eyeball movement measurement circuitry 13, an analysis circuitry 14, and an estimation circuitry 16. The athletic performance estimation apparatus 1 may include a normalization circuitry 15. The athletic performance estimation apparatus 1 performs processing of the steps illustrated in FIG. 3, thereby implementing the athletic performance estimation method of the first embodiment.

The athletic performance estimation apparatus 1 is, for example, a specialized apparatus configured by loading a specialized program into a known or dedicated computer that includes a central processing unit (CPU), a main memory (random access memory: RAM) and the like. The athletic performance estimation apparatus 1 executes processing steps under control of the central processing unit, for example. Data input into the athletic performance estimation apparatus 1 and data obtained by the processing steps are stored, for example, in the main memory; and the data stored in the main memory is read to the central processing unit as required so as to be used for other processing. The athletic performance estimation apparatus 1 may have at least a part thereof configured by hardware such as an integrated circuit. The athletic performance estimation apparatus 1 is, more specifically, an information processing apparatus with data processing functions, including a mobile terminal such as a smartphone or tablet, or a personal computer of desktop type or laptop type.

With reference to FIG. 3, procedures for the athletic performance estimation method executed by the athletic performance estimation apparatus 1 of the first embodiment will be described.

At step S11, the control circuitry 11 selects a measurement condition from among a plurality of preliminarily prepared measurement conditions. The control circuitry 11 outputs information indicating the selected measurement condition to the video presentation circuitry 12 and the eyeball movement measurement circuitry 13. The measurement condition is information for specifying a prediction task to be performed by an observed person. For example, as seen in the above experiment, for the task of predicting the direction in which a ball kicked by a kicker moves at the next time in a penalty kick scene in soccer, there are two kinds of preliminarily prepared measurement conditions: a “video in which the ball moves to the left” and a “video in which the ball moves to the right.” The kinds of measurement conditions may be appropriately set in view of the characteristics of a task to be executed, the skill level of an observed person, and the like; and the number of kinds thereof is not limited (that is, there may be three or more kinds thereof).

At step S12, the video presentation circuitry 12 displays, in accordance with information indicating a measurement condition which is input from the control circuitry 11, video corresponding to the measurement condition on an image presenting apparatus (not illustrated) such as a display. The video displayed is a video that includes a movement related to a target for a predetermined time period, which has been shot from the viewpoint of the observed person, and to be used for predicting the movement of the target at the time next to the predetermined time period. The target is an object that an observed person marks to observe the movement thereof, and may be a thing such as a ball or may be a person such as an opponent. For example, as seen in the experiment described above, the video of the scene in which a kicker runs for a ball placed at the center of a screen from a position on the right side of the center of the screen, which has been shot from the viewpoint of a goal keeper, will be displayed. Although, in the experiment described above, the predetermined time period started from about −200 milliseconds, the start time of the predetermined time period may be appropriately set in view of the characteristic of a task to be executed, the skill level of the observed person, and the like.

At step S13, the eyeball movement measurement circuitry 13 measures the movement of the eyes of the observed person at each time, which measurement is triggered by the input of the information indicating the measurement condition from the control circuitry 11. As the eyeball movement measurement circuitry 13, an existing device that measures the movement of eyes, such as an eye tracker, can be used. The eyeball movement measurement circuitry 13 associates time-series information on the measured eye movement with the information indicating the measurement condition which is input from the control circuitry 11; and outputs it to the analysis circuitry 14.

At step S14, the analysis circuitry 14 extracts the feature value of saccades (hereinafter, also referred to as “feature value based on eyeball movement”) from the time-series information on the movement of eyes which is input from the eyeball movement measurement circuitry 13. More specifically, the maximum angular velocity or maximum angular acceleration of the eyeball movement at each time is calculated from the time-series information on the movement of eyes; and time-series information including both a time when a result thereof exceeds a predetermined reference value (a time when microsaccades occur) and the amplitude thereof (the magnitude of microsaccades) is extracted as the feature value of saccades. The analysis circuitry 14 associates the extracted feature value of saccades with the information indicating the measurement condition which is input from the eyeball movement measurement circuitry 13; and outputs it to the normalization circuitry 15. In a case where the athletic performance estimation apparatus 1 does not include the normalization circuitry 15, the analysis circuitry 14 outputs the feature value of saccades to the estimation circuitry 16.

Processing of step S11 through step S14 is executed a plurality of times while changing the measurement condition randomly or in a predetermined order. On this occasion, it is preferable to control the measurement so that each preliminarily prepared measurement condition shall be executed almost the same number of times.

At step S15, the normalization circuitry 15 normalizes the feature value of saccades which is input from the analysis circuitry 14, in accordance with the measurement condition. More specifically, the feature values of saccades associated with other measurement conditions are converted so as to conform to a reference measurement condition that is selected from the plurality of preliminarily prepared measurement conditions. For example, in an example where two kinds of measurement conditions, the “video in which the ball moves to the left” and the “video in which the ball moves to the right,” are preliminarily prepared measurement conditions as in the experiment described above, when the “video in which the ball moves to the left” is selected as a reference measurement condition, the feature value of saccades associated with the “video in which the ball moves to the right” is integrated with the feature value of saccades associated with the “video in which the ball moves to the left” and averaging is performed, thereby obtaining the normalized feature value of saccades. The normalization circuitry 15 outputs the normalized feature value of saccades to the estimation circuitry 16.

At step S16, the estimation circuitry 16 calculates evaluation information of athletic performance based on the normalized feature value of saccades under the plurality of measurement conditions which is input from the normalization circuitry 15 (or the feature value of saccades under a plurality of measurement conditions which is input from the analysis circuitry 14); and outputs it. More specifically, it is determined whether there is a correlation between a time microsaccades occurred and the amplitude of microsaccades within a predetermined time period from the feature values of saccades under a plurality of measurement conditions; and outputs evaluation information corresponding to high athletic performance in the case there is a correlation or evaluation information corresponding to low athletic performance in the case there is not a correlation.

For example, the estimation circuitry 16 may calculate a correlation coefficient between a time microsaccades occurred and the amplitude of microsaccades within a predetermined time period and output a value of a predetermined monotonically increasing function in-the-broad-sense from the correlation coefficient absolute value, as evaluation information. The monotonically increasing function in-the-broad-sense “f” is a function that, if x<y, satisfies the relationship of f(x)≤f (y); “f” includes, although there may be a partial section in which an evaluation value does not change even when the correlation coefficient absolute value increases, at least a section where the evaluation value for larger correlation coefficient absolute value becomes larger than the evaluation value for smaller correlation coefficient absolute value; “f” is a function in which the above relation is never inverted throughout the range of values the evaluation value can take.

Alternatively, the estimation circuitry 16 may output binary evaluation information indicating, when the absolute value of the correlation coefficient is equal to or greater than a predetermined threshold, that athletic performance is high and indicating, when the absolute value of the correlation coefficient is smaller than a predetermined threshold, that athletic performance is low.

### Second Embodiment

In the first embodiment, the configuration is such that athletic performance is estimated by measuring the movement of the eyes of an observed person who is watching a video corresponding to a preliminarily prepared measurement condition. In a second embodiment, the configuration is such that athletic performance is estimated by measuring the movement of the eyes of an observed person in an actual sport environment instead of presenting video.

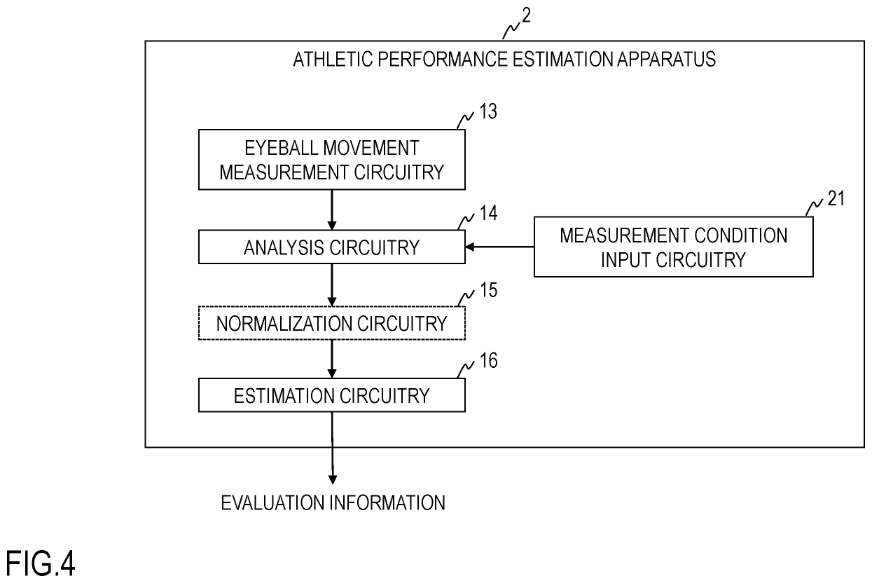

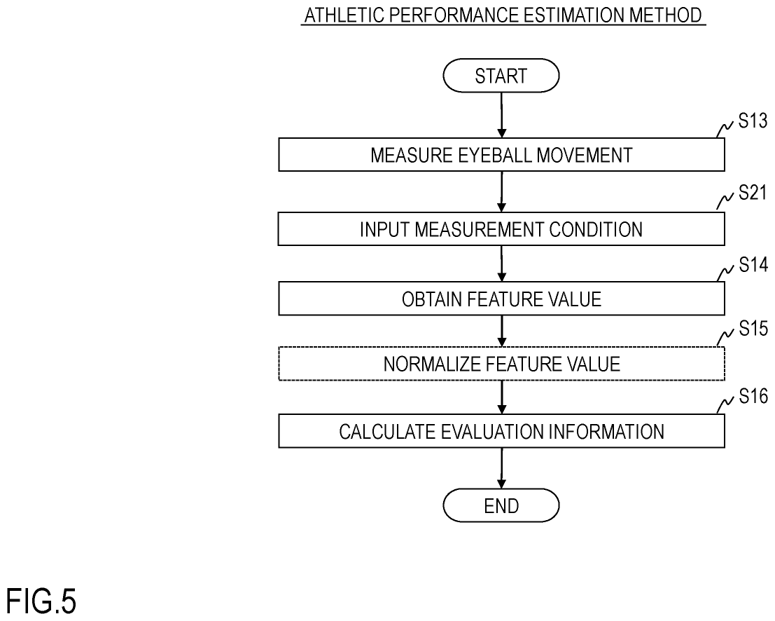

An athletic performance estimation apparatus 2 of the second embodiment includes, as illustrated in FIG. 4, for example, an eyeball movement measurement circuitry 13, an analysis circuitry 14, and an estimation circuitry 16, as with the first embodiment; and further includes a measurement condition input circuitry 21. The athletic performance estimation apparatus 2 may include a normalization circuitry 15, as with the first embodiment. The athletic performance estimation apparatus 2 performs processing of the steps illustrated in FIG. 5, thereby implementing an athletic performance estimation method of the second embodiment.

With reference to FIG. 5, procedures for the athletic performance estimation method executed by the athletic performance estimation apparatus 2 of the second embodiment will be described mainly regarding differences from the first embodiment.

At step S13, the eyeball movement measurement circuitry 13 measures the movement of the eyes of the observed person at each time in an actual sport environment. Since a timing when the target starts to move cannot be known in advance in an actual environment, the eyeball movement measurement circuitry 13 measures the movement of the eyes of the observed person all the time. The eyeball movement measurement circuitry 13 outputs time-series information on the measured movement of the eyes to the analysis circuitry 14.

At step S21, the measurement condition input circuitry 21 inputs information indicating a measurement condition (hereinafter, also referred to as “indication information”) from an input device such as a keyboard, touch panel, or mouse (not illustrated). For example, an observer other than the observed person observes an actual sport environment and inputs the direction in which a target has moved from an input device. For example, in the above experiment, she or he shall input, in a penalty kick scene in soccer, information indicating the direction in which the ball has moved by pressing a button (or key) that corresponds to the direction in which the ball kicked by a kicker has moved (the right direction or left direction from the viewpoint of the observed person). The measurement condition input circuitry 21 inputs the input indication information to the analysis circuitry 14, in association with the time-series information on the eye movement measured by the eyeball movement measurement circuitry 13.

At step S14, the analysis circuitry 14 extracts, each time the indication information is input from the measurement condition input circuitry 21, the feature value of saccades from the time-series information on the eye movement in a predetermined time period immediately before the time of the input. That is, the indication information that is input from the measurement condition input circuitry 21 corresponds to the information indicating a measurement condition used in the first embodiment. The analysis circuitry 14 outputs the extracted feature value of saccades to the normalization circuitry 15 or the estimation circuitry 16 in association with the indication information.

### Third Embodiment

In the first embodiment, the configuration is such that the normalized feature value of saccades is converted into evaluation information in accordance with a preliminarily provided correlation. In the third embodiment, the configuration is such that its implementation is achieved by a trained model that has learned the correlation by machine learning.

For example, the normalized feature value of saccades obtained from a plurality of skilled persons and the normalized feature value of saccades obtained from a plurality of unskilled persons are prepared as training data in advance, and a classifier is trained to classify, by using the normalized feature value of saccades as an input, it into a first group corresponding to skilled persons or into a second group corresponding to unskilled persons. For training of the classifier used here, a training method for a support vector machine (SVM) or another known identifier can be used.

The estimation circuitry 16 inputs the normalized feature value of saccades which is output from the normalization circuitry 15, to the trained classifier; obtains a classification result indicating that it corresponds to either the first group or the second group; and outputs it as evaluation information on athletic performance. The first group corresponds to being high athletic performance and the second category corresponds to being low athletic performance.

An input to a learner may be the feature value of saccades itself obtained from the observed person; not the normalized feature value of saccades. For example, a model can be so trained that the one obtained by associating a pair of feature values of microsaccades, which are obtained from each observed person under the two kinds of conditions of the case of “a ball kicked by a kicker moves to the left” and the case of “a ball kicked by a kicker moves to the right” for each observed person, with the correct answer of the performance of the observed person (the label of skilled/unskilled) is used as training data and evaluation information on athletic performance is output by using, as an input, a combination of the feature values of saccades. In this case, the configuration is such that instead of the normalization circuitry 15 and the estimation circuitry 16, a combination of the feature values of saccades which are output from the analysis circuitry 14 is input to the trained model and evaluation information on athletic performance is obtained.

Although the embodiments according to the present invention have been described above, the specific configurations are not limited to those embodiments and it is needless to say that appropriate design modifications or the like that are made without departing from the spirit of the present invention are included in the present invention. The various kinds of processing that has been described in the embodiments may be not only executed in time series in a description order but also executed in parallel or individually according to the processing capability of a device that executes the processing or according to the necessity.

[Program, Recording Medium]

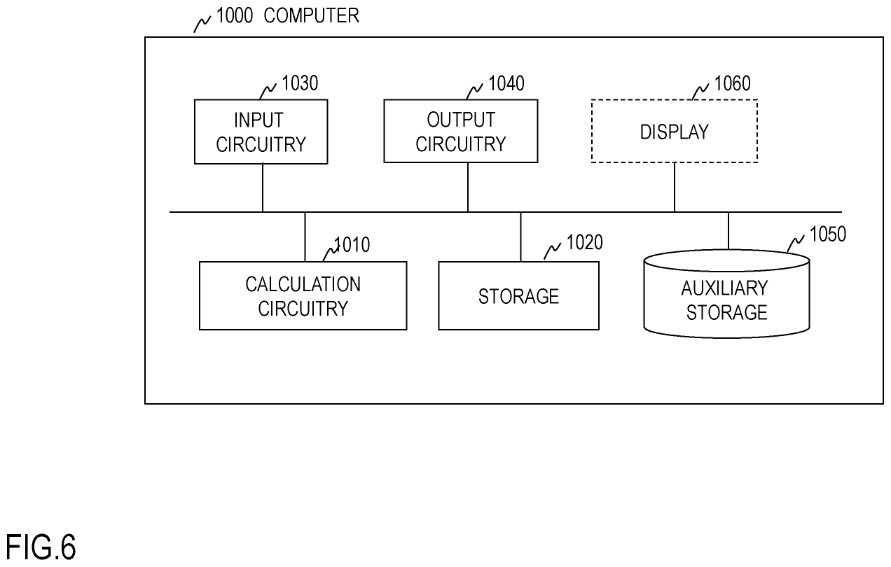

In a case where various processing functions in the devices described in the above embodiments are implemented by a computer, the processing contents of the functions to be included in the devices are described by a program. This program is loaded into a storage 1020 of a computer illustrated in FIG. 6 to cause a calculation circuitry 1010, an input circuitry 1030, an output circuitry 1040, and the like to operate, thereby various processing functions in the devices described above are implemented on the computer.

The program in which this processing contents are described can be recorded in a computer-readable recording medium. An example of the computer-readable recording medium is a non-transitory recording medium such as a magnetic recording device, an optical disk, or the like.

This program is distributed by, for example, selling, transferring, or lending a portable recording medium such as a DVD or a CD-ROM in which the program is recorded. Furthermore, a configuration may be adopted in which this program may be distributed by being stored in a storage device of a server computer and transferred from the server computer to another computer via a network.

A computer that executes such a program, for example, first temporarily stores either the program recorded in a portable recording medium or the program transferred from the server computer, in an auxiliary storage 1050 that is an own non-transitory storage device. In executing processing, this computer loads the program stored in the auxiliary storage 1050 that is an own non-transitory storage device, into the storage 1020 that is a transitory storage device; and executes processing according to the loaded program. As another mode for executing this program, a computer may load the program directly from a portable recording medium and execute processing according to the program; furthermore, each time a program is transferred from the server computer to this computer, may execute processing according to the received program. Moreover, a configuration may be adopted in which the above described processing is executed by a so-called application service provider (ASP) type service that implements processing functions only by execution instructions and result acquisition without transferring the program from the server computer to this computer. It should be noted that the program in the present mode includes information that is provided for processing by an electronic computer and conforms to a program (such as data that is not a direct command for the computer but has a property that defines the processing of the computer).

Furthermore, in this mode, the device is configured by causing a predetermined program to be executed on the computer; however, at least part of the processing contents may be implemented by hardware.

1. An athletic performance estimation apparatus comprising:
an analysis circuitry that obtains a feature value based on an eyeball movement of an observed person who is observing movement of a target; and
an estimation circuitry that estimates athletic performance of the observed person from the feature value obtained from the observed person, based on a predetermined relationship between the feature value based on the eyeball movement and a level of athletic performance.

2. The athletic performance estimation apparatus according to claim 1, wherein
the feature value is time-series information including a time when a saccadic eye movement occurs and an amplitude thereof; and
the estimation circuitry estimates the athletic performance of the observed person so that a case in which the time and the amplitude included in the feature value and within a predetermined time period immediately before the target moves has a correlation corresponds to a higher level of athletic performance than otherwise.

3. The athletic performance estimation apparatus according to claim 2, further comprising:
a classifier that has been trained in advance to classify the feature value obtained from the observed person into either a first category or a second category, the classification being performed using both the feature value obtained from a person falling under the first category and the feature value obtained from a person falling under the second category, the first category being of high athletic performance, the second category being of lower athletic performance than the first category,
wherein the estimation circuitry obtains a classification result as an estimation result of the athletic performance of the observed person, the classification result being obtained by inputting the feature value obtained from the observed person into the classifier.

4. The athletic performance estimation apparatus according to claim 2, wherein
the feature value is a normalized feature value obtained by integrating a first feature value with a second feature value, the first feature value being obtained from the observed person when the target moves in a first direction, the second feature value being obtained from the observed person when the target moves in a second direction different from the first direction.

5. An athletic performance estimation method, comprising:
an analysis circuitry obtaining a feature value based on eyeball movement of an observed person who is observing movement of a target; and
an estimation circuitry estimating athletic performance of the observed person from the feature value obtained from the observed person, based on a predetermined relationship between the feature value based on the eyeball movement and a level of athletic performance.

6. A non-transitory computer-readable recording medium which stores a program for causing a computer to function as the athletic performance estimation apparatus according to claim 1.
